(19) United States
(12) Patent Application Publication
(10) Pub. No.: US 2015/0093776 A1
(43) Pub. Date: Apr. 2, 2015

(54) POLYPHENOL, TERPENOID, GLYCOSIDE, AND ALKALOID PRODUCTION BY CROCUS SATIVUS CELL CULTURES

(71) Applicant: Dianaplantsciences, S.A.S., Rennes (FR)

(72) Inventors: Sung-Yong H. Yoon, Lake Oswego, OR (US); Raymond E. B. Ketchum, Tigard, OR (US); Colby G. Caldwell, Oregon City, OR (US)

(21) Appl. No.: 14/395,387

(22) PCT Filed: Apr. 19, 2013
(86) PCT No.: PCT/IB2013/001120

§ 371 Date: Oct. 17, 2014

Related U.S. Application Data
(60) Provisional application No. 61/635,639, filed on Apr. 19, 2012.

(51) Int. Cl. A01H 1/04; C12N 5/04; C12P 19/44; G01N 33/50; C12P 23/00; C12P 5/00

(52) U.S. Cl. CPC A01H 1/04; C12P 23/00; C12P 5/007; C12P 19/44; G01N 33/5097; C12N 5/04
USPC 435/34; 435/67; 435/166; 435/74; 435/410

(57) ABSTRACT

A method for producing metabolites of Crocus sativus (C. sativus) includes (i) selecting a cell line of C. sativus that produces one or more saffron metabolites in cell suspension culture, and (ii) growing the selected cell line in a suspension cell culture to produce the saffron metabolite.

### BACKGROUND

Saffron is a spice derived from the flower of Crocus sativus, commonly known as the saffron crocus. Each saffron crocus grows to 20 to 30 cm (8 to 12 in) and bears up to four flowers, each with three vivid crimson stigmas at the distal end of a carpel (Rashed-Mohassel 2006). Saffron crops flower for 30 to 40 days in the autumn with each plant flowering for up to 15 days (Deo 2003). The stigmas are collected by hand, dried, and used in various cuisines as a seasoning and coloring agents. It takes about 150,000 to 200,000 flowers and over 400 hours of hand labor to produce 1 kg of dried saffron (Deo 2003). Because each flower's stigmas need to be collected by hand, and there are only a few stigmas per flower, saffron is the most expensive spice in the world. In August 2011, SaffronExporter.com quoted prices of US $1400 to $1850 per kilogram of Iranian saffron, depending on the grade.

Estimates of the world's total production of dried saffron are highly variable depending on the source of the estimate and the year of production, from around 50 tons per year as cited by Oberdieck (1991) to 205 tons per year as cited by Ahmad et al. (2011). According to UN ComTrade statistics, total saffron exports in 2011 totaled nearly $373 million dollars. At $1500 per kilogram, that would equate to worldwide saffron production in 2011 of about 250 tons.

Crocus sativus L. is a small geophyte, cultivated worldwide and known as a source of the spice saffron that is used for cooking, staining, medicine, cosmetics and some other purposes. Saffron (Crocus sativus L.) is a triploid (2n=3x=24), sterile plant and a member of the Iridaceae family, which includes about 60 genera and 1,500 species (Caiola and Canini 2010). The plants in this family are herbaceous with underground storage organs such as rhizomes, corms or bulbs. The Crocus genus includes approximately 85 species worldwide, but only about 30 species are cultivated (Rasheed-Hohassel 2006).

Saffron's corms are covered by tunics and consist of nodes and are internally made up of starch-containing parenchyma cells. A corm survives for only one season, reproducing via division into “cormlets” that eventually give rise to new plants. Each corm produces five to eleven leaves. The photosynthetic activity of the leaves during the early winter and the early spring months contribute to the formation of the replacement corms at the base of the shoots. In autumn, each corm produces 1 to 4 deep violet to purple fragrant flowers with dark veins and a darker violet color in the throat. The style is divided into three deep red clavate branches, each branch being 25 to 32 mm long. Much of the style exceeds the anthers and at least half the length of the perianth. The style arises at a point well below the base of the anthers in the throat of the flower (Rasheed-Hohassel 2006). The three-branch style of C. sativus flowers is the most economically important part of the saffron plant.

Saffron is believed to contain over 150 volatile and non-volatile compounds, but only about one-third of those constituents have been isolated and characterized (Abdullaev 2002). Compounds considered pharmacologically active and important are volatile agents (e.g., safranal), bitter principles (e.g., picrocrocin) and dye materials (e.g., crocetin and its glycoside, crocin) (Abdullaev 2002).

Saffron produces an unusual class of carotenoids, the water-soluble C20 apocarotenoid, crocetin (8,8′-diapo-8,8′-carotenedioic acid) and its ester derivatives and glycosides, collectively referred to as crocins (Pfander and Schurtenburger 1982). Crocins are the most characteristic components of saffron stigmas because they are responsible for their distinctive color. The most important and abundant of these crocins is α-crocin, the trans-crocetin di-(β-D-gentiobiosyl) ester. Crocin imparts a rich golden-yellow hue to foods and is also used as a textile dye. Other compounds present in saffron include fat-soluble carotenoids such as lycopene, α- and β-carotene and zeaxanthin.

Saffron's bitter taste and fragrance result primarily from the chemicals picrocrocin, a result of the oxidation of zeaxanthin (Raina et al. 1996), and safranal, produced by deglycosylation of picrocrocin through heating or action of β-glucosidase (Himeno and Sano 1987). Saffron quality, regulated by ISO 3632-2:2010 (Spices—Saffron (Crocus sativus L.)—Part 2: Test methods), is determined spectrophotometrically by measuring bitterness (picrocrocin @ 257 nm), aroma (safranal @ 330 nm) and coloring strength (crocin @ 440 nm).

Saffron and saffron extracts have also been used in traditional medicine to treat various conditions (for a recent review see Wani et al. 2011). For example, saffron has been used to treat nervine, melancholia and hysteria, depression, cramps, asthma, cough and bronchospasms, as an expectorant, menstruation disorders (amenorrhea, dysmenorrhea, leucorrhea), for soothing the gastrointestinal tract in dyspeptic disorders, as a carminative, for fever, liver damage, anemia, rheumatism, neuralgia, toothache, septic inflammations, as a supportive treatment of various forms of cancer, e.g. abdominal tumors, cancers of the bladder, ears, kidneys, liver, neck, spleen, stomach, breast, mouth and uterus, as a stimulant and aphrodisiac, for stimulation of circulation, and for prevention of premature ejaculation (Wani et al. 2011).

A number of effects of saffron extracts were recently examined in pharmacological test models, including anti-tumor effects, cell-protective effects, reduced toxicity of cytostatics, improvement of learning abilities and memory, anti-depressive effects, anti-Parkinson effects, antioxidant effects, anti-inflammatory effects, blood lipid lowering effects, promoting effects on wound healing, effects on hear and circulation, spasmolytic effects, effect on the immune system, effects on blood clotting and anti-allergic effects (Wani et al. 2011). In addition, it is now known that crocins have powerful antioxidant properties and the ability to quench free radicals, which may be useful in cancer treatment and prevention (Ochiai et al. 2004).

Commercial utilization of saffron's chemical components (e.g., terpenoids, carotenoids, apocarotenoids, monoterpenes, terpenoids, and the like) has been limited due to its high price and relatively restricted availability. Plant cell culture provides an attractive alternative source of saffron metabolites to the harvesting of stigmas by hand from field grown plants during the limited time each fall when the plants flower.

### BRIEF DESCRIPTION OF THE DRAWINGS

To further clarify the above and other advantages and features of the present disclosure, a more particular description of the subject matter of the disclosure will be rendered by reference to specific embodiments thereof which are illustrated in the appended drawings. It is appreciated that these drawings depict only illustrated embodiments of the disclosure and are therefore not to be considered limiting of its scope. The subject matter of the disclosure will be described and explained with additional specificity and detail through the use of the accompanying drawings in which:

FIG. 1 shows a simplified depiction of the apocarotenoid pathway from 4-carotene to crocetin, picrocrocin, and saffranal;

FIG. 2 shows a total UV absorption chromatogram of saffron extract and a total mass chromatogram from the same injection, where the multitude of peaks in the total ion chromatogram indicates a complex mixture of metabolites in the extract;

FIG. 3 is a comparison of UV chromatograms of crocin in saffron extract and a saffron standard measured at 443 nm, the crocin peak is easily identifiable at 16.4 minutes;

FIG. 4 is a comparison of UV chromatograms of safronal in saffron extract and a safronal standard measured at 312 nm, the safranal peak is identifiable at 29.2 minutes; and

FIG. 5 is a plot of 3 chromatograms monitored at different UV wavelengths from a single injection of a methanolic saffron extract.

### DETAILED DESCRIPTION

Because saffron (C. sativus) is a triploid hybrid, and therefore sterile, propagation is entirely vegetative relying on separation and planting of corms that have developed from the previous season's parent corms. Much of the recent scientific research on saffron has related to developing alternative methods to classical breeding (see Ahmad et al (2011) and Ascuogh et al (2009) for recent reviews), and has included in vitro micropropagation (Dhar and Sapru 1993; Ding et al. 1979; George et al. 1992; Homes et al 1987; Ilahi et al 1987; Sano et al 1987; Sheibani et al. 2007; Wani and Mohiddin et al. 2009) and attempts at organogenesis of stigmas and stigma-like structures as a means of producing saffron in vitro (Fakhrai and Evans 1990; Han and Zhang 1987; Himeno and Sano 1987; Luskotov et al. 1999; Mir et al. 2010; Sarma et al. 1990). Plant cell cultures of de-differentiated C. sativus cells are an alternative to producing important compounds from C. sativus plants.

To produce crocin derivatives in relatively high quantities there is a need to develop a sustainable in vitro culture system for Crocus sativus cells. While there has been some research into the production of natural products from C. sativus in vitro, that work has been limited to working with callus cultures on solid medium (Chen et al. 2003). Embodiments of the invention disclosed herein utilize a liquid suspension culture, which allows sufficient scaling to produce commercially viable quantities of these natural products.

The present disclosure relates to cells of Crocus sativus that are configured to grow as suspension cell cultures in a liquid medium. The cells are derived from one or more C. sativus plant parts, such as a floral parts (e.g., petals, ovary, anthers and stigmas), stem, leaf, corm, or root. The cells are adapted to grow to a high density within a selected period of time (e.g., greater than 5, 10, or 15 days and/or less than 30, 25, or 20 days and/or within a range of the foregoing days).

In addition, the cells may be grown under specific cell culture conditions (e.g., growth medium, light conditions, production medium, with the addition of other compounds) that allow the cells to produce high concentrations of one or more selected secondary metabolite compounds, including, but not limited to, terpenoids, polyphenols, glycosides, and alkaloids. In particular, the cells are grown under specific cell culture conditions that allow the cells to produce high concentrations of selected carotenoids and/or apocarotenoids, terpenes and/or terpenoids, and glycosides thereof.

As used herein, the term “secondary metabolites” are organic compounds produced by an organism that are not necessary for the plant to go through a complete life-cycle. Unlike primary metabolites, absence of secondary metabolites does not result in immediate death, but rather in long-term impairment of the plant's survivability, fecundity, or aesthetics, or perhaps in no significant change at all (Fraenkel 1959). Secondary metabolites often play an important role in plant defense against herbivory and other interspecies defenses. Humans tend to use plant secondary metabolites as medicines, flavorings, and recreational drugs.

There are a number of related biosynthetic pathways in Crocus sativus that give rise to classes of secondary metabolites that are the subject of the present disclosure. These include compounds derived from isoprene and include the terpene, carotenoid, and apocarotenoid biosynthetic pathways.

The terpenoids (sometimes also referred to as isoprenoids) are a large and diverse class of compounds derived from five-carbon isoprene units assembled and modified in thousands of ways (Gershenzon and Dudareva 2007). Many are multicyclic structures that differ from one another not only in functional groups but also in their basic carbon skeletons. These compounds can be found in all classes of living things, and are the largest group of natural products. Plant terpenoids are used extensively for their aromatic qualities—saffron is no exception. Plant terpenoids play a role in traditional herbal remedies and are under investigation for antibacterial, antineoplastic, and other pharmaceutical functions (Gershenzon and Dudareva 2007).

Carotenoids are tetraterpenoid organic pigments that naturally occur in the chloroplasts and chromoplasts of plants and some other photosynthetic organisms. There are over 600 known carotenoids, which are split into two classes: xanthophylls (which contain oxygen) and carotenes (which are purely hydrocarbons, and contain no oxygen). Carotenoids are light harvesting pigments and absorb blue light particularly strongly (450 to 480 nm). They serve two key roles in plants and algae: they absorb light energy for use in photosynthesis, and they protect chlorophyll from damage from photooxidation (Demmig-Adams and Adams 1996).

Apocarotenoids are organic compounds that occur widely in living organisms. Apocarotenoids are derived from carotenoids by oxidative cleavage, catalyzed by carotenoid oxygenases. Examples include crocetin, which is one of the primary coloring compounds of saffron; the vitamin A retinoids retinal, retinoic acid, and retinol; and the plant hormone abscisic acid.

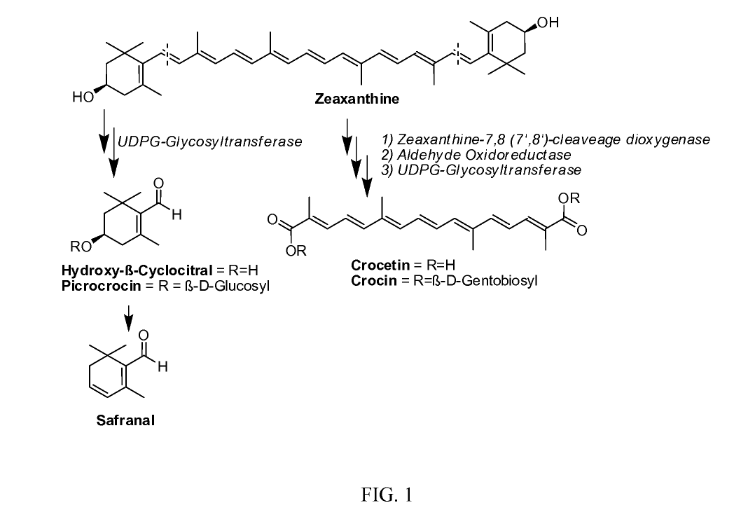

The major compounds of saffron that are currently considered to be economically, culinarily, and pharmaceutically valuable include, but are not limited to, crocetin, the crocins (i.e., crocin, different enantiomers of crocin, and the like), picrocrocin, and safranal. While the color of saffron is mainly due to the degraded carotenoids (crocin and crocetin), the flavor comes from the carotenoid oxidation products (mainly safranal and the bitter glucoside picrocrocin). It was proposed by Pfander & Schurtenberger (1982) that the biogenesis of the coloring compounds and the volatile flavoring compounds is derived by bio-oxidative cleavage of zeaxanthin. The proposed biosynthetic pathway is illustrated in FIG. 1 (from Gomez-Gomez et al. 2010).

In one embodiment, the present disclosure relates to cell lines derived from Crocus sativus cells and methods for making the cell lines. The cells can be grown in suspended cell culture to produce high concentrations of saffron metabolites such as selected carotenoids and/or apocarotenoids, terpenes and/or terpenoids, and glycosides of these. For example, cultures may be selected to produce high concentrations of one or more of a mixture of metabolites of crocetin, crocin and other crocins, the monoterpene aldehyde picrocrocin, or safranal.

In another embodiment, the present disclosure is concerned with identifying specific cell culture conditions (e.g., growth and/or production medium formulation, environmental conditions, addition of compounds to stimulate metabolite formation) that allow C. sativus cell cultures to produce high concentrations of one or more carotenoids and/or apocarotenoids, terpenes and/or terpenoids, and glycosides thereof. For example, cell culture conditions may be selected to allow the isolated cells to produce high concentrations of one or more of a mixture of metabolites consisting of crocetin, crocin and other crocins, the monoterpene aldehyde picrocrocin, or safranal. The cell suspension cultures may be grown from cells that produce at least 0.7% by weight of the saffron metabolites (i.e., crocetin, crocin and other crocins, the monoterpene aldehyde picrocrocin, or safranal) in the cell suspension culture (on a dry weight basis). In other embodiments, the percentage of the foregoing saffron metabolites may be at least 0.7%, 1.0%, 2.0% 5.0%, 10%, 20% and/or less than 95%, 80%, 50%, 30%, 20%, or within a range of the foregoing percentages. In some embodiments, the cell line may be selected based on its production of particular saffron metabolites. For example, the cell line can be selected for its ability to produce certain combinations of the foregoing saffron metabolites, such as, but not limited to a combination of crocetin and crocin in the foregoing concentrations and/or ranges.

An elite C. sativus cell line that is capable of producing high concentrations of one or more of a mixture of metabolites consisting of crocetin, crocin and other crocins, the monoterpene aldehyde picrocrocin, or safranal may be identified by one or more methods, such as visual inspection, a fluorimetric method, measuring the optical properties of the culture medium, monitoring rates of growth and rates of carbon source consumption, and any method of chromatographic separation and detection of mixtures metabolites from cells, media, and cell and media extracts.

In some embodiments, measuring the optical properties of spent culture medium is used to determine growth and productivity. For example, periodic measurements of Brix, a refractive measurement of the solute concentration of a liquid, is used to determine how rapidly cultures are growing as a function of their rate of carbohydrate consumption. Likewise, substances in growth media or that may be secreted into media may be monitored by optical density (“OD”) to gauge the rate of growth of cells. For example, many common constituents of growth media (e.g., vitamins) have characteristic spectra and absorbances and their rate of consumption can be used to estimate the rate of growth of the culture. Many metabolites of saffron also have characteristic spectra and may be excreted into the medium in detectable quantities in rapidly growing cultures. In one example, spent medium can be inspected for the presence of crocins or other desirable metabolites of saffron. Cell lines that are identified by their optical density (OD) to be rapidly-growing and high-producing can be more rigorously analyzed by HPLC or LC-MS to identify all of the constituents of the cells.

In some embodiments, a plurality of cell lines are grown or cultivated and individual cell lines that produce desired metabolites or desired concentrations of certain metabolites may be analyzed and compared to one another. Analyzing the constituents of the plurality of different cell lines allows certain cell lines to be selected that produce the desired metabolites in the desired concentrations and/or have particularly desired characteristics such as a desired growth rate.

In yet another embodiment, the present disclosure describes the identification of medium compositions for growth and production for both primary and multi-stage cultures. The term “multi-stage culture” describes culturing methods that include an early stage or stages where biomass is increased and a later stage or stages where production of the desired metabolites is initiated or enhanced. The different stages may be defined by a change of medium from the early stage to the later and/or by the addition of a compound or mixture of compounds that will stimulate the production of desired metabolites.

In yet another embodiment, the present disclosure describes the identification of light conditions (wavelength, intensity, duration, and/or cycle) for production of crocetin, picrocrocin, safranal, or other crocins, or conversion of crocin to picrocrocin. The growth of photosynthetic organisms is affected by light, particularly the ability to convert carbon dioxide and water into carbohydrates. For plants grown in culture, this parameter is usually not a relevant concern because the nutrient needs of the cells are provided by the growth medium. Nevertheless, light conditions can affect key developmental stages, growth of cells, and the production of metabolites. For example, crocin production can be stimulated by exposure to UV light. In one embodiment, a cell suspension medium is exposed to UV light to stimulate production of desired metabolites. The wavelength of the UV light may be in a range from 250-400 nm. The intensity of the light may be 0 (dark) or greater than 500, 1000, or 5000 lux and less than 20,000, 10,000, or 5,000, or within a range thereof. The duration of the UV light may be continuous or intermittent.

Other growth conditions for producing desired concentrations of saffron metabolites include a temperature of at least 15, 20, or 22° C. and/or less than 30, 25, or 23° C. or within a range of the foregoing temperatures. The cell suspension culture may be grown in the dark or under various lighting conditions. The lighting of the cell suspension culture may be selected to be a white light (e.g., wavelength of 380 to 700 nm) and a lux of at least 500, 1000, 5,000, 10,000, or 20,000, and/or a lux less than 100,000, 50,000, or 25,000 or within a range of the foregoing lux. The lighting may be continuous or cyclical. Light cycles can have a duration of at least 6, 8, 10, or 12 hours and/or less than 24, 20, or 16 hours or within a range thereof. The light cycles of light and dark may be symmetrical (e.g., 12 hrs dark/12 hrs light) or asymmetrical (e.g., 8 hrs dark/16 hrs light or 16 hrs dark, 8 hrs light).

The cell suspension culture may be grown without agitation or agitated using a rate of at least 50, 100, or 200 rpm and/or less than 500, 300, or 200 rpm or within a range thereof. The cell suspension culture may also be aerated. Aeration can be with ambient air, or air mixed with pure oxygen. The rate of aeration may be at least 10, 25, or 50, or 100 L/hr and/or less than 500, 100, or 50 L/hr, or within a range thereof.

In yet another embodiment, the present disclosure describes the identification of a compound or mixture of compounds that stimulates the production of crocetin, picrocrocin, safranal, or other crocins, or conversion of crocin to picrocrocin in a cell suspension. Examples of such compounds include, but are not limited to jasmonic and salicylic acid and their esters, heavy metals, triazoles, chitin, and the like.

Growing the cells in a cell suspension culture allows the metabolites to be produced in higher concentrations as compared to traditional native plant production or even on solid medium. These higher concentrations can be achieved by selecting growth conditions that promote production of particular secondary metabolites with minimal detrimental affects on the cells in suspension culture. Suitable growth conditions for cell suspension cultures has been found to differ substantially from optimal growth conditions for the native plant, thereby allowing the selection of growth conditions that stimulates production of desired metabolites without the same detrimental consequences that would be observed from such growth condition for the native plant. In some cases, conditions that are applied to the cell suspension culture are not practical to apply to a native plant because of the differences in cultivating a plant in suspension medium verses growing a plant in soil. Or alternatively, the concentrations or intensities of the treatments in cell suspension cultures can be lower than the concentrations needed to invoke the desired response when applied to the native plant.

The present invention also relates to large-scale synthesis of saffron metabolites using cell suspension cultures. In this embodiment, a cell line selected to produce one or more saffron metabolites at a desired rate or concentration is selected (e.g., a higher rate or concentration as compared to the metabolite production in the native plant). The cell line is then cultivated in a suspension cell culture under conditions suitable to produce the metabolite at the desired rate or concentration. The bioreactor may have a volume of at least 1, 10, 100, 500, or 1,000 liters. The large-scale synthesis may include producing an inoculum and adding the inoculum to the bioreactor. In some embodiments the inoculum may have at least 5, 10, 25, or 50 grams of fresh cell weight/liter (“gFCW/L”) and/or less than 500, 250, 150, and/or 100 gFCW/L, or have a concentration within a range of any of the foregoing concentrations.

The vessel used to culture the suspension cells (inoculum or production culture) may be a glass flask, wave bag, bubble-type bioreactor, stir-type bioreactor or the like in a configuration from 1:1 to 5:1 vertical:horizontal geometry.

In still yet another embodiment, the present disclosure describes extraction methods for improving extraction efficiency or selectivity between crocin and picrocrocin.

The subject matter of the disclosure will be described and explained with additional specificity and detail through the use of the following Examples.

### EXAMPLES

### Example 1

### Surface sterilization of Corms

Crocus sativus corms were washed in tap water with or without detergent, washed with distilled or deionized water, dipped in 50 to 95% ethanol for 1 to 60 s, surface sterilized for 1 to 60 min in 0.1 to 3% sodium hypochlorite solution with or without a surfactant and then rinsed with sterile distilled or deionized water.

The apical buds were excised from the corms and were dissected. The floral parts were removed and separated into petals, ovary, anthers and stigmas. The stigmas were then separated into two parts by being cut just below the point at which the pigmentation develops. The non-pigmented part between the ovary and the pigmented part was also kept. Explants were dissected into pieces of 0.5 to 1 cm.

Corms were cut horizontally into 3 to 4 slices, each of which was further cut into segments with and without an axillary bud. The elongated corms were dissected to expose floral and vegetative primordia, each about 0.5 to 1 cm long.

### Example 2

### Callus Induction of Crocus sativus

Dissected explants were inoculated on custom or established media formulations, for example MS medium (Murashige and Skoog 1962), LS medium (Linsmaier and Skoog 1965), B5 medium (Gamborg et al. 1968), White medium (White 1943) or N6 medium (Nitsch and Nitsch 1969) containing a carbohydrate source such as sucrose, glucose, maltose, lactose, and/or fructose at a total concentration of 1 to 10% carbohydrate. Furthermore, the media contained plant growth regulators in concentrations varying from 0 to 20 mg/L. Auxins may include: 4-chlorophenoxyacetic acid, 2,4-dichlorophenoxyacetic acid, 2,4,5-trichloroacetic acid, naphthaleneacetic acid, indoleacetic acid, indolebutyric acid, triiodobenzoic acid, β-naphthoxyacetic acid (NOA), phenyl acetic acid, and picloram. Cytokinins may include adenine and it salts, kinetin, isomers of zeatin and its riboside, benzylaminopurine, 2-isopentenyl adenine, 1,3-diphenylurea (DPU), N-(2-Chloro-4-pyridyl)-N-phenylurea (4-CPPU), and thidiazuron[1-Phenyl-3-(1,2,3-thiadiazol-5-yl)urea]. Other plant growth regulators may include dicamba, abscissic acid, giberellic acid and one or more of its isomers, paclobutrazol, ancymidol, jasmonic acid and its esters, phloroglucinol, chlorocholine chloride, N-(phosphonomethyl)glycine (glyphosate), and succinic acid 2,2-dimethylhydrazide. Additional components may be added to the medium to enhance growth and productivity including, but not limited to, banana powder, yeast extract, coconut water, protein hydrolysates, amino acids, vitamins, and activated charcoal. All media were solidified by 0.1 to 1.5% agar or other suitable gelling material after adjusting the pH of 5.25 to 6 and autoclaved for time sufficient to insure sterilization at 1.2 kg/cm². Cultures were incubated at 15 to 30° C. in dark, in continuous light, or any combination of varying light and dark conditions. After 1 to 8 weeks, calli could be observed originating from the explants.

An example of a typical, though not necessarily the optimal medium is B5NB solidified with 0.8% agar (Ketchum and Gibson 1995), however other media provided effective results.

Callus derived from C. sativus tissue was subcultured every 1 to 8 weeks on predefined medium. Cell lines were selected for further proliferation and maintenance from callus that was a yellow to dark golden to red color, friable, and grew rapidly.

### Example 3

### Elite Callus Line Screening of Crocus sativus

Carotenoids are very characteristic and important components of Crocus sativus, responsible for the distinctive colors of this plant. This characteristic was used for elite callus line selection on solid plates. In order to select appropriate callus lines for carotenoid and apocarotenoid production, calli were first screened visually according to callus friability, growth and color. Carotenoids like zeaxanthin and β-carotene have yellow to orange color and apocarotenoids like crocin have a distinctive orange to red color, which were easily distinguishable in the calli. Red, yellow and gold-colored calli were selected since they contain high concentrations of crocin and its biosynthetic precursors. However, some colored calli had a tendency to brown and the browning was found to spread to unpigmented calli. Careful selection of non-browning calli was also a selection criterion.

The callus plates selected visually were screened by fluorescence observation under violet-blue light excitation. At 435 to 440 nm excitation crocin exhibits a distinctive bright yellow-green fluorescence. Spectroscopic studies on crocin in solution at pH 10 showed an absorption maximum at 410 to 430 nm and strong emission peak at 543 nm under excitation of 436 nm. At pH 4 to 6 on solid plates fluorescence was not as strong as at pH 10, but the screening method using fluorescence was effective to select calli producing high concentrations of crocin. Samples from the calli selected by fluorescence were analyzed by LC-MS in order to confirm presence of the targeted crocins and other metabolites.

### Example 4

### Saffron Analysis by HPLC with PDA and ESI-MS Detection

An HPLC-PDA-MS method was developed to separate and detect the major metabolites of saffron. This method is applicable to extracts of saffron plants as well as to saffron plant cells in culture. An example of a suitable method is to use a reversed-phase C-18 column and a water-acetonitrile mobile-phase gradient to separate the components of saffron extracts. A PDA-spectrometer and an ESI tandem mass analyzer were used to detect and characterize the components of the extract as they elute from the column.

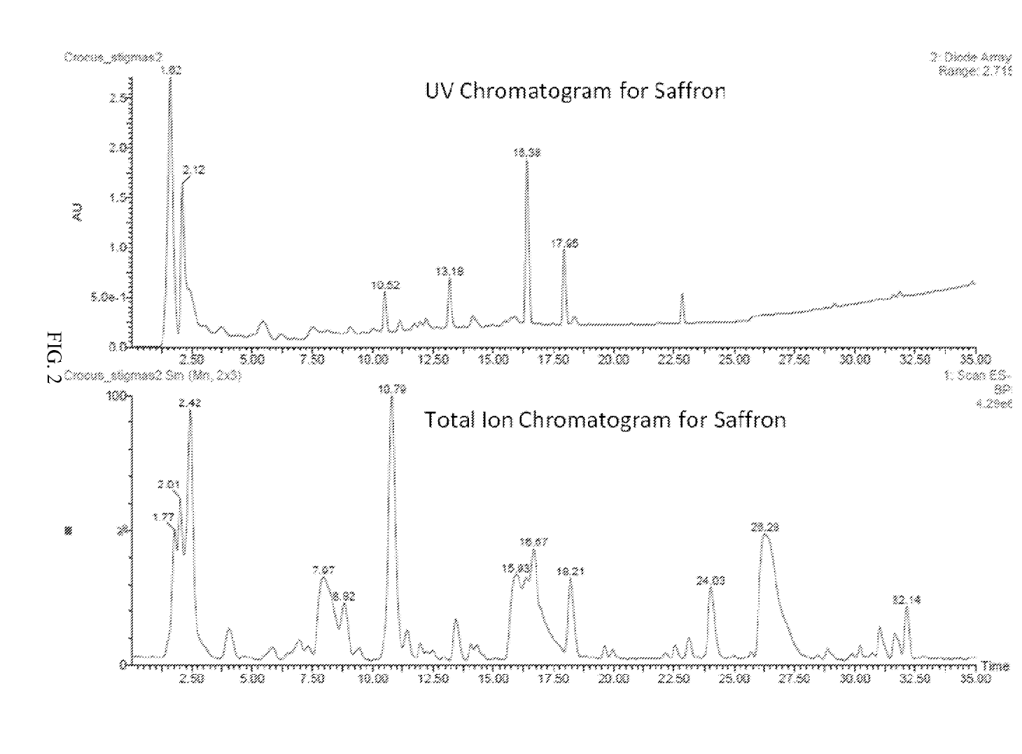

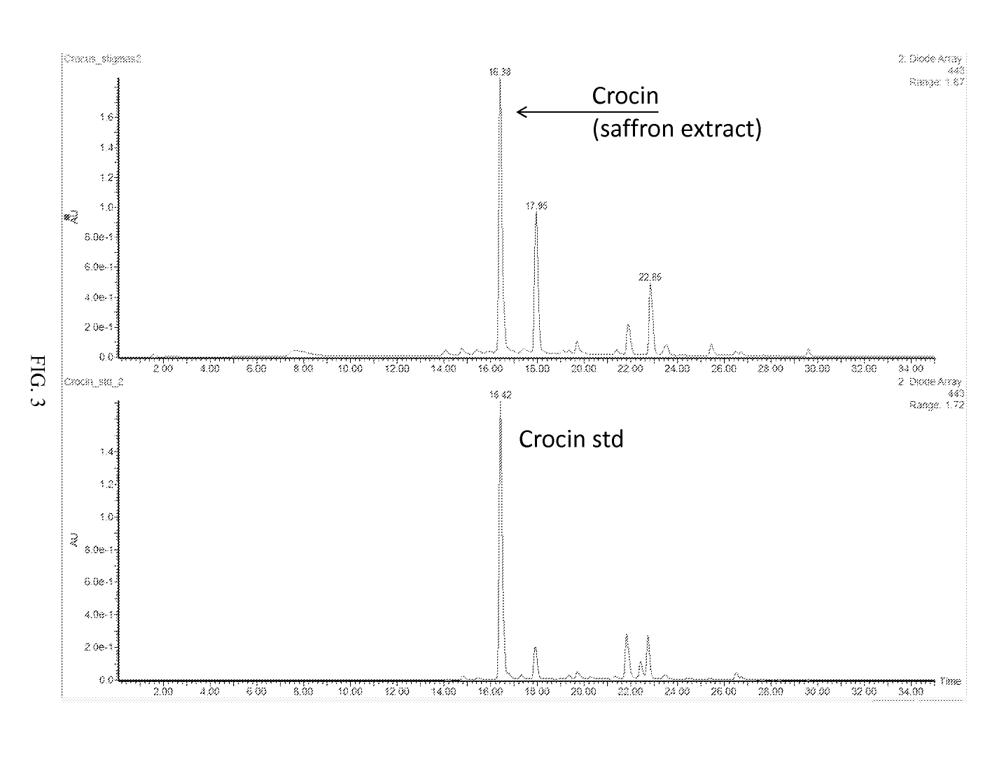

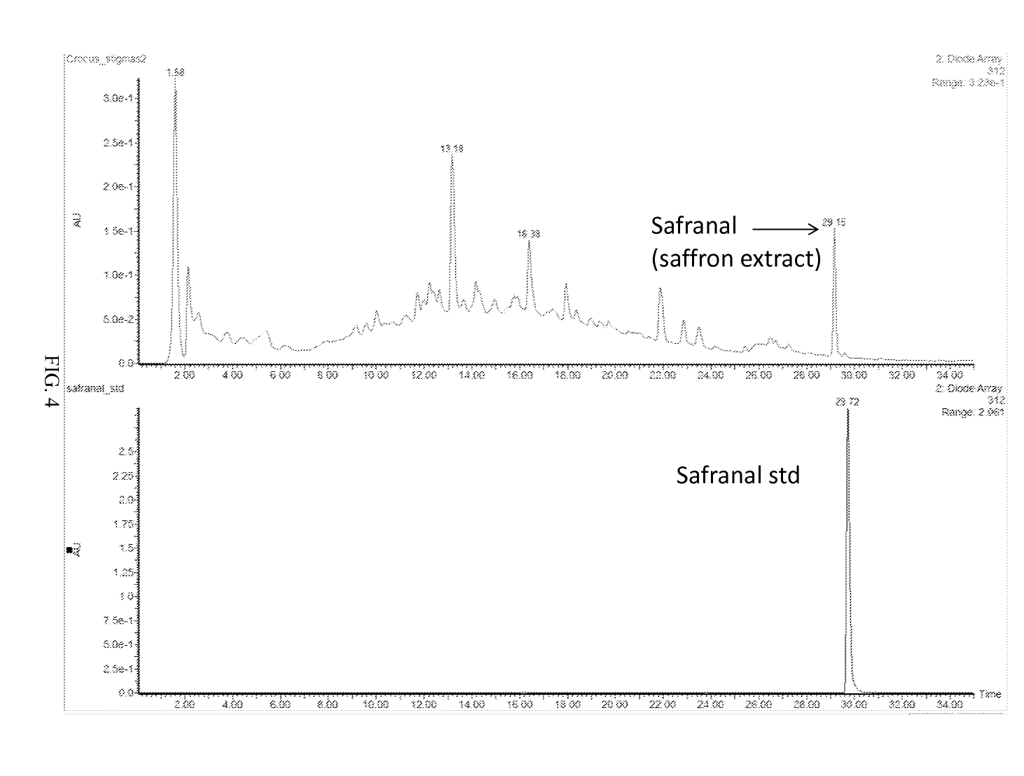

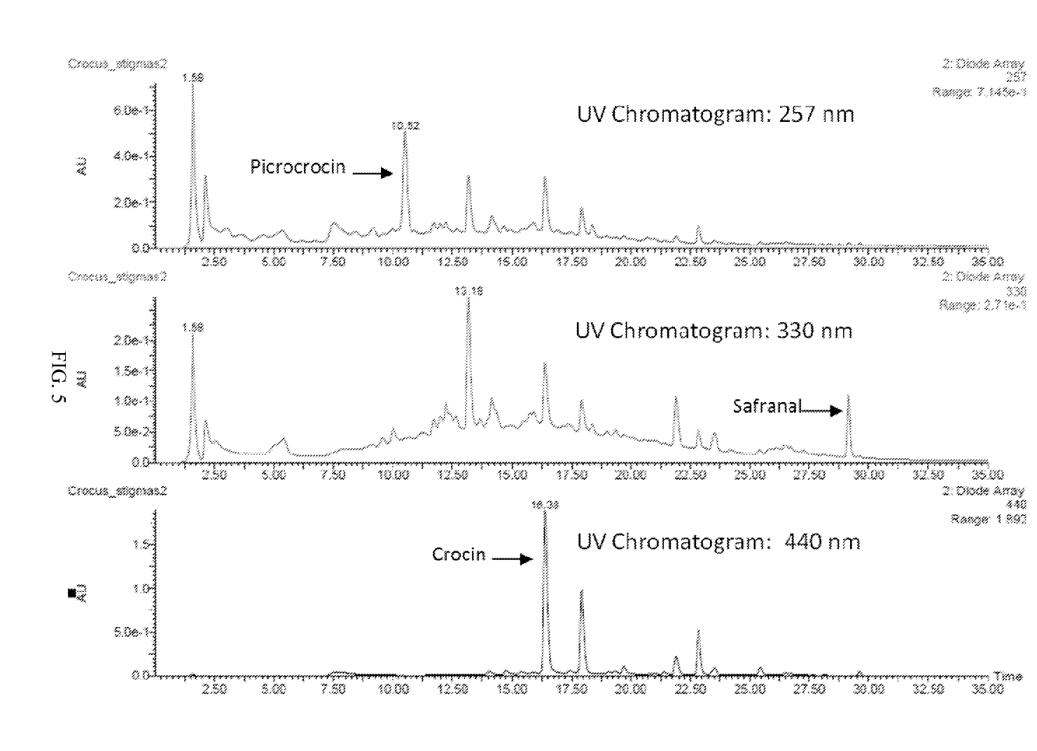

While this method was general enough to detect a wide range of metabolites (FIG. 2), specific attention was focused on detection and quantification of 3 high-value saffron compounds: crocin, picrocrocin and safranal. These compounds were identified by their UV and mass spectra and by comparison with authentic standards (FIGS. 3-4). Calibration curves built from these standards were used for quantification. Mass spectra were acquired in negative ion mode from 140 to 1200 amu, and UV chromatograms were also monitored at 3 different wavelengths corresponding to the absorption maxima of picrocrocin (257 nm), safranal (330 nm) and crocin (440 nm), (FIG. 5).

### Example 5

### High-Throughput Spectrophotometric Determination of Total Crocins in Crocus Cell Cultures

The following describes a high-throughput method of extraction and spectrophotometric analysis for determination of total crocins content in crocus callus or suspension cells. Standards of authentic crocin (Sigma) were prepared at 2.5, 5, 10, 50, 100, and 200 μg/mL in 70% methanol. Extracts were made by adding 2 mL of 70% methanol to 100 mg of fresh cells (1:20 extraction ratio) in a 2 mL Eppendorf tube. To homogenize the mixtures a tungsten bead was added to each tube, the tubes were capped, placed in 24 slot sample racks and shaken for 4 minutes at 18 Hz on a Mixer Mill 300 (Retsch). Up to 48 samples may be processed simultaneously in this way. The extracts were centrifuged at 4000 rpm for 4 minutes, then 200 μl of the clear supernatant from each tube was transferred to a well on a UV-plate. 200 μl of each standard dilution were added to separate wells in the same plate. Absorbances of the extracts and the standards were measured at 440 nm. Content of total crocins in the extracts was determined against a standard curve made from the crocin standards.

### Example 6

### Suspension Creation from Callus Derived from Crocus sativus

Friable and golden-colored cell lines were chosen for initiation of suspensions. Cell suspensions were created by introducing C. sativus callus (prepared as in Example 2 and 3) into liquid medium in sterile Erlenmeyer flasks. The medium used in this Example is the medium that was found in Example 2 to have provided the best combination of rapid callus growth and high metabolite production. An example of this type of medium is B5NB (Ketchum and Gibson 1995). The flasks were covered with sterile silicone (foam) caps and agitated at 50 to 150 revolutions per minute (rpm) in a gyrorotary shaker. The suspensions were kept in darkness at 15 to 30° C. To establish the cell culture, the spent medium was removed and fresh medium was added. The growth of cells was measured by the rate of carbohydrate consumed by measuring the delta of ° Brix of the medium. If the ° Brix was less than or equal to half of the initial value of the medium, fresh medium was added to the cells. If the ° Brix was greater than half, fresh medium was only added after 2 weeks.

### Example 7

### Optimization of Cell Growth

This example describes methods used to increase cell growth of suspensions. Volumetric productivity of the target compounds increases as a function of the rate of cell growth and the final biomass at which cell growth stops. To determine the optimal inoculum size, suspension cultures of Crocus sativus cells were initiated with an inoculum size of 5 to 100 gram of fresh cell weight/liter (“gFCW/L”) and grown for 14 days in various medium conditions. Growth regulators, basal medium salts, carbohydrate sources and organic/inorganic sources were tested for maximizing final biomass for the culture period.

As an example of fresh weight inoculation, cultures initiated at a cell density of 25 gFCW/L did not reach maximal density within 7 days. Final biomass initiated at a cell density of 50 gFCW/L increased more than five times in biomass (gFCW/L) within 14 days and some cultures grew to 270 to 300 gFCW/L by day 14. Cell selection resulted in cultures that reached more than 250 gFCW/L within 14 days or less (a rapidly growing cell culture). Cultures that took more than 14 days to reach 200 gFCW/L were discarded.

For the 50 gFCW/L inoculum, a doubling time of 5.62 days results in a calculated final biomass accumulation of 280 gFCW/L by day 14. In comparison, a 100 gFCW/L inoculum size with a doubling time of 8.03 days reaches 335 gFCW/L by day 14; however the color of the cells was darker brown than the culture with a lower inoculation density. Optical density (OD) at day 14 was used for selection of well-grown flasks. A hand-held portable OD scanner was used to measure biomass as non-invasive indirect method. Calibration curve between OD values of spent medium or culture and FCW was constructed in advance and OD values at day 14 indicated final biomass with the unit of gFCW/L.

### Example 8

### Cell Selection of Crocin High-Producing C. sativus Suspension Culture

Carbohydrate consumption is an indicator of culture growth and metabolite production. Refractive index of suspension cultures are monitored throughout the culture cycle. Cultures that had not completely exhausted carbohydrates by day 14 were considered to be undesirable and discarded.

Crocin production was measured by three methods: spectrophotometrically, by LC-MS analysis, and by CIELAB color measurement. After cells settled, CIELAB CIE 1976 (L*, a*, b*) color space data values of the settled cells were measured by a hand-held, portable spectrophotometer. This gave a “Lab” value for the of crocin color of the settled cells. Only the top 10 to 15% of high-crocin producing flasks selected by their “Lab” values were sampled for LC-MS analysis. The cell selection process, where flasks that produced higher than average intracellular crocin content were selected as quantified by a high production level of crocin at each subculture, allowed for further improvement in crocetin, crocin, picrocrocin and safranal production levels.

### Example 9

### Optimization of Crocin Production from C. sativus Suspensions by Multi-Stage Culture

Cell growth and crocin production were separated by using a multi-stage culture process. In the first stage, biomass production was maximized. In the second stage, production of desired secondary metabolites (e.g., crocetin, crocin, picrocrocin, and safranal) was maximized. The multi-stage culture method was found to be the best way of maximizing biomass and crocin production by replacing the growth medium at the end of the growth cycle with production medium. Because of the challenges of completely draining and replacing the medium in a large-scale bioreactor process, the production medium formulation had the same basal formulation of salts and vitamins as the growth medium, but contained additional modifications such as different types or concentrations of carbohydrates, plant growth regulators, metabolic elicitors, additional salts or vitamins, or other components that enhance the production of saffron metabolites.

Light requirement for crocetin, crocin, picrocrocin and safranal production was also studied. Their PY (production yield, mg product/gDCW cells) values were tested by statistical design of experiments (DOE) by comparing different light intensities, wavelengths and exposure times and the best conditions were used for the second culture stage.

### Example 10

### Elicitor Development for Overproduction of Apocarotenoids and Terpenes

Several compounds and mixtures of compounds were tested for production of crocetin, crocin, picrocrocin, and safranal production. Typical examples of these types of compounds include, but are not limited to, jasmonic and salicylic acid and their esters, heavy metals, triazoles, chitin, and the like. Different concentrations and combinations of these compounds were added into the optimized production medium at day 14. The experiment was carried out using randomized complete block design. The FCW was determined at day 21. Thereafter the cells were freeze-dried to constant weight for determination of dried cell weight (DCW). Dried samples were used for further LC-MS analysis to determine crocetin, crocin, picrocrocin, safranal content in the dried cells.

### Example 11

### Extraction of Apocarotenoids and Terpenes from C. sativus Suspension Cultures

This example describes methods developed for extracting apocarotenoids and terpenes from suspension cells of C. sativus cultures developed in examples 1 to 8. C. sativus fresh cells without media or dried and ground C. sativus cells were resuspended in ethanol:water (50:50, v/v), stirred for 1 to 3 hr at room temperature in dark. The suspension was centrifuged at 3000 to 5000×g for 20 min to separate cell residue and the supernatant was collected. The same process was repeated with the cell residue for improving extraction efficiency. The solid phase was discarded and the supernatant was used for analysis or further purification processes.

For LC-MS analysis, the supernatant was centrifuged for 4 minutes at 6000×g (RCF 5996) and filtered with 0.45 μm membrane filter and diluted 10-fold by using the same aqueous extraction solvents prior to analysis. The remaining extracts were stored in −20 degree of freezer for further analyses.

### Example 12

### Scale-Up of C. sativus Suspension Culture

A common problem in the use of plant cell cultures is obtaining consistent production of target products (Kim et al., Biotechnol Prog. 20(6) 1666, 2004). Therefore, a key for successful large-scale plant cell culture is to maintain stable productivity. A process to scale-up suspensions of C. sativus cell cultures from 125 mL flasks to 250 mL and then 500 mL flasks was successfully conducted in a medium determined to be optimal in example 5, such as B5NB (Ketchum and Gibson 1995). The speed of the shakers was optimized for 500 mL flasks to give the same kind of growth and production numbers as in the 125 mL flasks. Different shaker speeds were tested—50 to 150 RPM and their DO (dissolved oxygen) concentrations were compared to growth in 125 mL flasks. The agitation speed and working volume was adjusted to provide similar DO concentrations in both 125 mL flasks and 500 mL flasks. The final biomass was 350 to 400 gFCW/L, which was about 7 times greater than the initial biomass of 50 gFCW/L for all of the treatments. The effects of agitation speed and culture volume on production yield (PY) and biomass was also studied. Biomass, sugar concentration in medium, conductivity, and metabolite productivity were measured at regular intervals during the culture cycle.

Experiments to scale up cultures to grow in larger containers (e.g. 2.8 L Fernbach flasks) were conducted and agitation speed (rpm), shaker stroke size and working volume was optimized based on DO concentratuion trend, final biomass and PY of crocetin, crocin, picrocrocin and safranal. This successfully yielded similar growth and production as in the smaller 125 mL and 500 mL flasks.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for producing metabolites of Crocus sativus (C. sativus), comprising:
selecting a cell line of C. sativus that produces one or more saffron metabolites selected from carotenoids, apocarotenoids, terpenes, terpenoids, glycosides of these, and combinations of these; and
growing the selected cell line in a suspension cell culture to produce the one or more saffron metabolites,
wherein the one or more saffron metabolites produced is at least 0.7% by weight of cells of the suspension cell culture on a dry weight basis.

2. (canceled)

3. The method of claim 1, wherein the one or more saffron metabolites produced is at least 2.0% by weight of the cells of the suspension cell culture on a dry weight basis.

4. The method of claim 1, wherein the one or more saffron metabolites produced is at least 10.0% by weight of the cells of the suspension cell culture on a dry weight basis.

5. The method of claim 1, wherein at least 1% of cells of the suspension culture on a dry weight basis includes one or more saffron metabolites selected from crocetin, crocin, picrocrocin, and/or safranal, and combinations thereof.

6. The method of claim 1, wherein growing the selected cell line includes exposing the cell suspension culture to UV light.

7. The method of claim 1, wherein the suspension cell culture is grown in a multi-stage culture, an early stage or stages comprising a medium that produces a desired rate of cell growth and a later stage or stages comprising a medium that increases production of carotenoids and/or apocarotenoids, terpenes and/or terpenoids, and glycosides thereof.

8. The method of claim 1, wherein the cell suspension culture is grown in a bioreactor.

9. The method of claim 8, wherein the bioreactor has a volume of at least 1 liter.

10. The method of claim 8, wherein the bioreactor is inoculated with an inoculum having a density in a range from 5-500 gram of fresh cell weight/liter (“gFCW/L”).

11. The method of claim 8, wherein a growth rate of the cells causes the suspension cell culture to reach a maximum density in less than 30 days.

12. The method of claim 1, further comprising:
cultivating a plurality of cell lines of C. sativus; and
determining the concentration of the one or more saffron metabolites produced in each of the cell lines, wherein the selected cell line is selected at least in part on the concentration of the one or more saffron metabolites.

13. The method of claim 12, wherein the basis for selecting the cell line includes a result obtained through:
visual observation;
fluorimetric analysis;
a chromatographic technique, including but not limited to paper, column, thin-layer, liquid, and/or a tandem chromatography techniques, including but not limited to HPLC-MSⁿ, UPLC-MSⁿ, UPLC-NMR, and/or UPLC-MS-NMR;
monitoring growth of the cell lines directly or indirectly;
monitoring carbon source metabolism or catabolism;
monitoring oxygen and carbon dioxide utilization or production; and/or
monitoring the pH of the culture medium.

14. The method of claim 13, wherein the cell line is selected based in part on the selected cell line having a yellow, gold, orange, or red color as compared to cell lines not selected.

15. The method of claim 12, wherein the concentration of the one or more saffron metabolites in the selected cell line is at least 0.7%, 1.0%, 2.0% 5.0%, 10%, or 20% by weight of the cells of the selected cell line on a dry weight basis.

16.-20. (canceled)

21. The method of claim 1, wherein the cells of the suspension cell culture are adapted to reach more than 250 gFCW/L within 14 days or less.

22. The method of claim 1, wherein the selected cell line comprises callus cells derived from one or more C. sativus plant parts.

23. An isolated cell line of C. sativus having a desired production of a metabolite, the cell line being produced by the method of claim 1.

24. An isolated cell line of C. sativus having a desired production of a metabolite, the cell line comprising:
a plurality of cells derived from one or more C. sativus plant parts, the cells being adapted to produce one or more saffron metabolites selected from carotenoids, apocarotenoids, terpenes, terpenoids, glycosides of these, and combinations of these in suspension cell culture, wherein the one or more saffron metabolites produced is at least 0.7%, 1.0%, 2.0% 5.0%, 10%, or 20% by weight of the cells in the suspension cell culture on a dry weight basis.

25. An isolated cell line as in claim 24, wherein the cells comprise one or more of:
callus derived from C. sativus tissue;
cells adapted to grow to a density greater than 250 gFCW/L within 14 days or less in the suspension cell culture;
one or more saffron metabolites at a concentration of at least 0.7%, 1.0%, 2.0% 5.0%, 10%, or 20% by weight of the cells on a dry weight basis;
cells having a yellow, gold, orange, or red color; and
cells adapted to grow at a growth rate sufficient to cause the suspension cell culture to reach a maximum density in less than 30 days.

26. An isolated cell line as in claim 24, wherein the cell line is selected to produce at least 0.7%, 1.0%, 2.0%, 10%, or 20% crocetin, crocin, picrocrocin, and/or safranal in cell suspension culture based on dry weight.
